(19) United States
(12) Patent Application Publication
(10) Pub. No.: US 2018/0199831 A1
(43) Pub. Date: Jul. 19, 2018

(54) BLOOD PRESSURE MEASUREMENT DEVICE

(71) Applicants: DENSO CORPORATION, Kariya-city, Aichi-pref. (JP); A&D COMPANY, LIMITED, Tokyo (JP)

(72) Inventors: Taiji KAWACHI, Kariya-city (JP); Kouki FUTATSUYAMA, Kariya-city (JP); Hiroshi YAMAKITA, Kariya-city (JP); Yasuhiko SHINOZAKI, Tokyo (JP); Toshiaki MACHITANI, Tokyo (JP)

(73) Assignees: DENSO CORPORATION, Kariya-city, Aichi-pref. (JP); A&D COMPANY, LIMITED, Tokyo (JP)

(21) Appl. No.: 15/744,498

(22) PCT Filed: Sep. 5, 2016
(86) PCT No.: PCT/JP2016/075934

§ 371 Date: Jan. 12, 2018

(30) Foreign Application Priority Data
Oct. 9, 2015 (JP) 2015-201218

(51) Int. Cl. A61B 5/021; A61B 5/022; A61B 5/00

(52) U.S. Cl. CPC A61B 5/02108; A61B 5/02141; A61B 5/02416; A61B 5/704; A61B 5/7207; A61B 5/022

(57) ABSTRACT

A pulse wave sensor is to detect a pulse wave signal from a measurement target finger, which is a part of fingers of a subject. A placement table is to enable a palm of the subject to be placed thereon. A blood pressure measurement unit is to measure a blood pressure based on the pulse wave signal detected with the pulse wave sensor. An upper surface of the placement table includes a palm placement region that has a shape, which is convex upward with respect to a longitudinal direction of the palm and is inclined to lower on a side of a little finger in a lateral direction of the palm.

### CROSS REFERENCE TO RELATED APPLICATION

This application is based on Japanese Patent Application No. 2015-201218 filed on Oct. 9, 2015, the disclosure of which is incorporated herein by reference.

### TECHNICAL FIELD

The present disclosure relates to a blood pressure measurement device.

### BACKGROUND ART

Conventionally, a method for acquiring a pulse wave signal from a finger of a subject by using a pulse wave sensor and for measuring a blood pressure based on the pulse wave signal has been known (refer to Patent Literature 1).

### PRIOR TECHNICAL LITERATURE

### Patent Literature

PATENT LITERATURE 1: Publication of unexamined Japanese patent application No. 2002-172094

When acquiring a pulse wave signal, in a case where an unnecessary force is applied to a finger of a subject, noise in the pulse wave signal becomes large. In this case, a blood pressure cannot be accurately measured.

### SUMMARY OF INVENTION

It is an object of the present disclosure to provide a blood pressure measurement device that enables to reduce noise in a pulse wave signal.

According to one aspect of the present disclosure, a blood pressure measurement device comprises a pulse wave sensor to detect a pulse wave signal from a measurement target finger, which is a part of fingers of a subject. The blood pressure measurement device further comprises a placement table to enable a palm of the subject to be placed thereon. The blood pressure measurement device further comprises a blood pressure measurement unit to measure a blood pressure based on the pulse wave signal detected with the pulse wave sensor. An upper surface of the placement table has a palm placement region in a shape, which is convex upward with respect to a longitudinal direction of the palm and which is inclined to lower on a side of a little finger in a lateral direction of the palm.

### BRIEF DESCRIPTION OF THE DRAWINGS

The above and other objects, features and advantages of the present disclosure will become more apparent from the following detailed description made with reference to the accompanying drawings. In the drawings:

The aforementioned object, other objects, characteristics, and advantages of the present disclosure become more apparent from a description that will be given with reference to the accompanying drawings. The drawings are as follows:

FIG. 1 is a block diagram illustrating a configuration of a blood pressure measurement device;

FIG. 2 is a perspective view illustrating a configuration of a blood pressure measurement device;

FIG. 3 is a plan view illustrating a configuration of the blood pressure measurement device;

FIG. 4 is a side view illustrating a configuration of the blood pressure measurement device when viewed from a first side;

FIG. 5 is a side view illustrating a configuration of the blood pressure measurement device when viewed from a second side;

FIG. 6 is a side view illustrating a configuration of the blood pressure measurement device when viewed from a third side;

FIG. 7 is a cross-sectional view taken along a line VII-VII in FIG. 3;

FIG. 8 is a perspective view illustrating a state in which a palm is placed on a housing of the blood pressure measurement device;

FIG. 9 is a cross-sectional view illustrating the state in which the palm is placed on the housing of the blood pressure measurement device;

FIG. 10 is a perspective view illustrating a configuration of the blood pressure measurement device;

FIG. 11 is a graph illustrating a pulse wave signal acquired using the blood pressure measurement device;

FIG. 12 is a graph illustrating a pulse wave signal acquired using the blood pressure measurement device;

FIG. 13 is a side cross-sectional view illustrating another configuration of an upper surface;

FIG. 14 is a side view illustrating another configuration of the blood pressure measurement device when viewed from a side of a side 41;

FIG. 15 is a cross-sectional view illustrating a cross section of a blood pressure measurement device of another configuration similarly to FIG. 7; and

FIG. 16 is a cross-sectional view illustrating a state in which a palm is placed on a housing of a blood pressure measurement device according to another configuration.

### DESCRIPTION OF EMBODIMENTS

An embodiment of the present disclosure will be described with reference to the drawings.

### First Embodiment

1. Configuration of a Blood Pressure Measurement Device 1

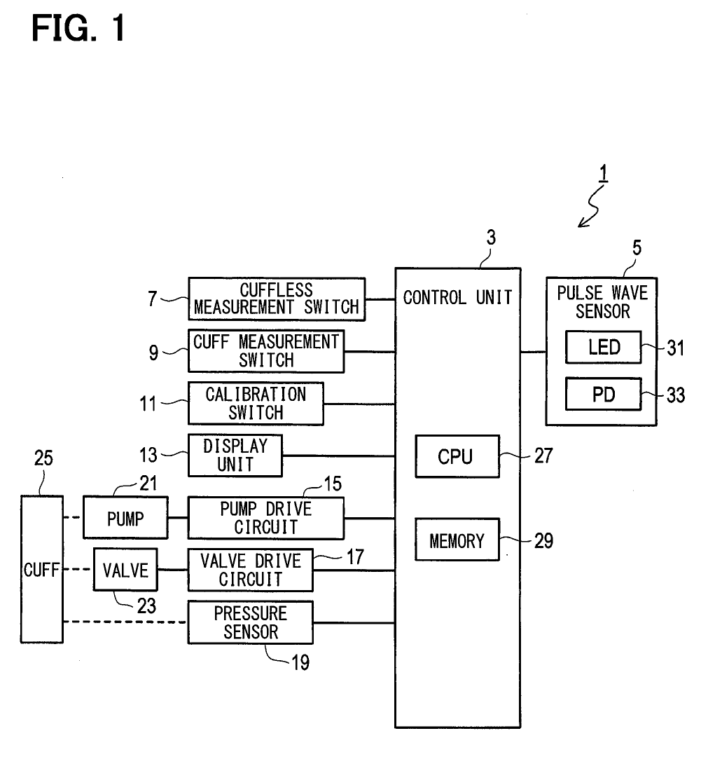

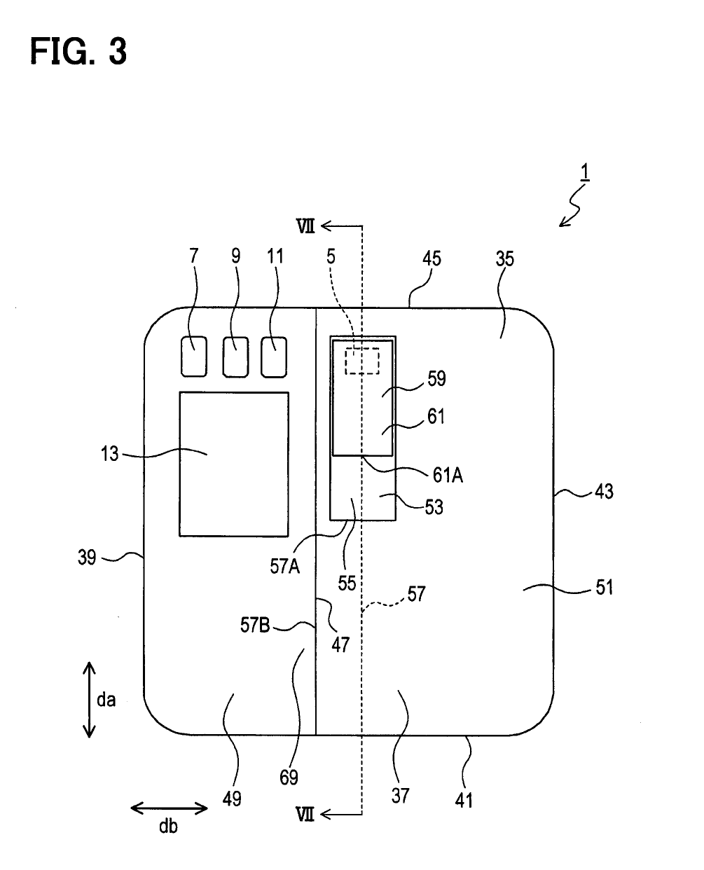

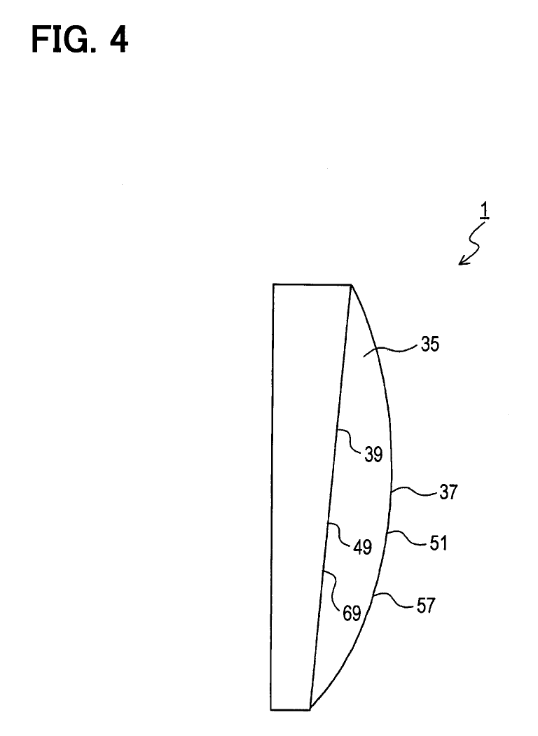

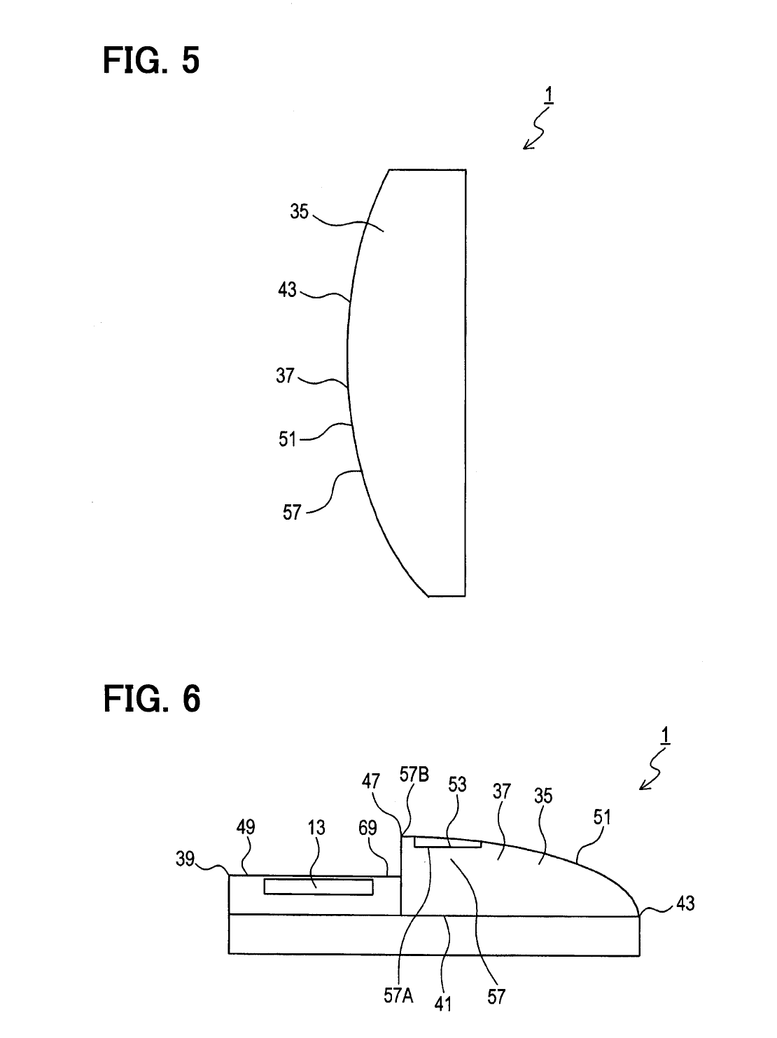

A configuration of a blood pressure measurement device 1 will be described with reference to FIGS. 1 to 9. As illustrated in FIG. 1, the blood pressure measurement device 1 includes a control unit 3, a pulse wave sensor 5, a cuffless measurement switch 7, a cuff measurement switch 9, a calibration switch 11, a display unit 13, a pump drive circuit 15, a valve drive circuit 17, a pressure sensor 19, a pump 21, a valve 23, and a cuff 25.

The control unit 3 is mainly configured with a well-known microcomputer having a CPU 27 and a semiconductor memory such as a RAM, a ROM, a flash memory (hereinafter referred to as a memory 29). The various functions of the control unit 3 are produced by causing the CPU 27 to execute a program stored in a non-transitory tangible recording medium. In this example, the memory 29 corresponds to the non-transitory tangible recording medium storing the programs. In addition, methods corresponding to the programs are implemented by the execution of the programs. The number of microcomputers configuring the control unit 3 may be one or more.

The control unit 3 corresponds to a blood pressure measurement unit. Further, a technique for realizing the functions of the control unit 3 is not limited to software, but a part or all of the functions may be produced by using hardware combined with a logic circuit, an analog circuit, or the like.

The pulse wave sensor 5 is a well-known sensor capable of acquiring a pulse wave signal from a finger of a subject. The finger from which the pulse wave sensor 5 acquires the pulse wave signal is hereinafter referred to as a measurement target finger. In the present embodiment, the measurement target finger is an index finger of a right hand. The pulse wave sensor 5 includes a light emitting diode (LED) 31 and a photodiode (PD) 33. The light emitting diode 31 irradiates a skin of the measurement target finger with a visible light. The wavelength of the light is 5000 Å to 7000 Å. A part of the light irradiated by the light emitting diode 31 is reflected in a capillary blood vessel of the skin. The photodiode 33 receives the light reflected in the capillary blood vessel in the light irradiated by the light emitting diode 31 and takes out the received light as an electric signal. The extracted electric signal is a pulse wave signal that fluctuates reflecting the pulse wave of the subject.

The cuffless measurement switch 7, the cuff measurement switch 9, and the calibration switch 11 are each a user operable switch. When the cuffless measurement switch 7 is operated, the blood pressure measurement device 1 implements a cuffless measurement to be described later. When the cuff measurement switch 9 is operated, the blood pressure measurement device 1 implements a cuff measurement to be described later. The cuff measurement is a well-known blood pressure measurement using the cuff 25. When the calibration switch 11 is operated, the blood pressure measurement device 1 implements a calibration process to be described later. The display unit 13 is a display capable of indicating images.

The pump drive circuit 15, the valve drive circuit 17, the pressure sensor 19, the pump 21, the valve 23, and the cuff 25 are configured to implement the cuff measurement. The cuff 25 is structured to accommodate a rubber bag in a cloth band-like bag. The cuff 25 is wound around an arm on a side opposite to an arm to which the measurement target finger belongs in subject's arms. For example, when the measurement target finger is the index finger of the right hand, the cuff 25 is wound around the left arm of the subject.

The pressure sensor 19 detects a pressure in the cuff 25. The pump drive circuit 15, the valve drive circuit 17, the pump 21, and the valve 23 control the pressure in the cuff 25. In other words, the pump 21 is driven by the pump drive circuit 15, the valve 23 is opened with the valve drive circuit 17, and an air is introduced into the cuff 25, thereby being capable of increasing the pressure inside the cuff 25. Further, a state of the valve 23 is switched to another with the valve drive circuit 17 and the air is pulled out from the cuff 25, thereby being capable of reducing the pressure inside the cuff 25.

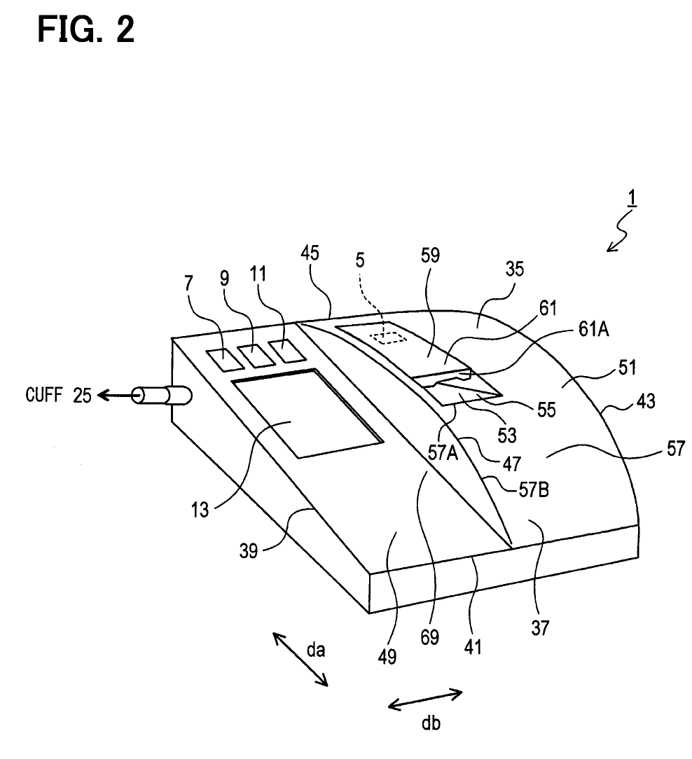

As shown in FIGS. 2 to 9, the blood pressure measurement device 1 includes a housing 35. The housing 35 corresponds to a placement table. The housing 35 is in a box shape. An upper surface 37 of the housing 35 is in a rectangular shape when viewed from above. The respective four sides of the upper surface 37 are assumed to be sides 39, 41, 43, and 45. Hereinafter, when viewed from above, a direction parallel to the sides 39 and 43 is defined as a longitudinal direction da. In addition, when viewed from above, a direction orthogonal to the longitudinal direction da is defined as a lateral direction db. The longitudinal direction da and the lateral direction db are directions in a horizontal plane.

The upper surface 37 is divided into a first region 49 and a second region 51 by a boundary line 47 parallel to the longitudinal direction da. The first region 49 has a planar shape. The cuffless measurement switch 7, the cuff measurement switch 9, the calibration switch 11, and the display unit 13 are provided in the first region 49. The first region 49 is inclined so that the first region 49 on the side of the side 41 becomes lower. The display unit 13 is located so as not to overlap with a thumb placement region 69 to be described later.

The second region 51 has a curved surface shape excluding a finger fitting groove 53 to be described later. The curved surface shape in the second region 51 is configured as follows. In other words, as shown in FIGS. 2 and 4 to 9, the second region 51 is convex upward with respect to the longitudinal direction da. A shape convex upward with respect to the longitudinal direction da represents that the second region 51 is convex upward in a cross section along a vertical plane parallel to the longitudinal direction da. An example of the upward convex shape includes a mountain-shaped curved surface shape. Further, as shown in FIG. 6, the second region 51 is inclined so that the second region 51 on the side of the boundary line 47 is higher, and the second region 51 on the side of the side 43 is lower in the lateral direction db.

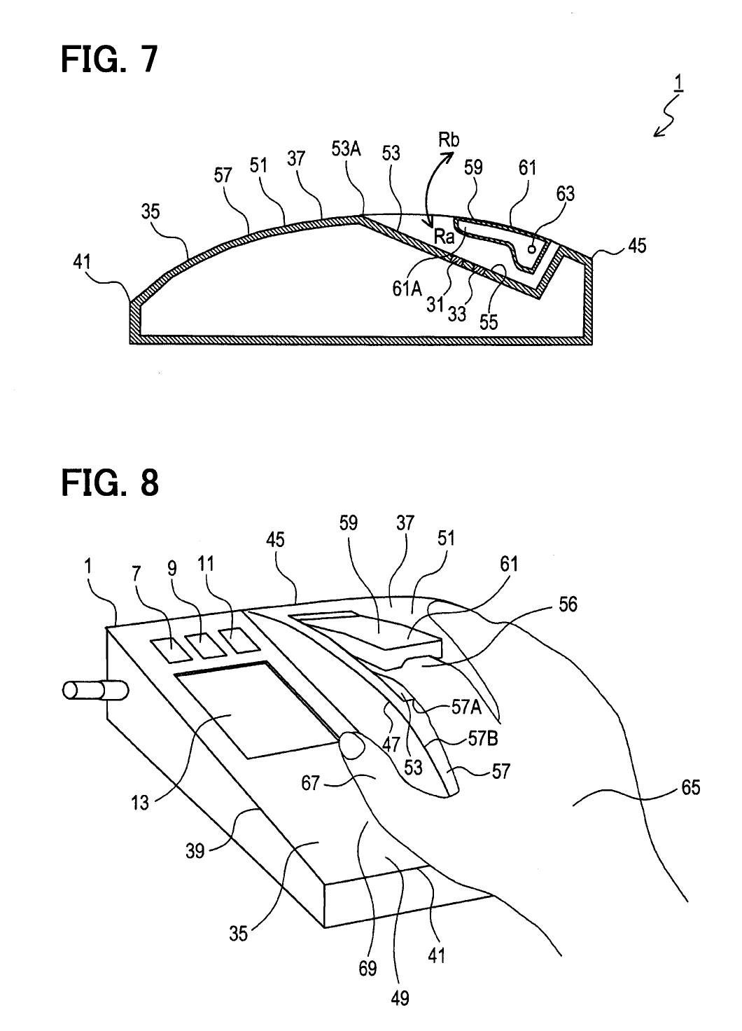

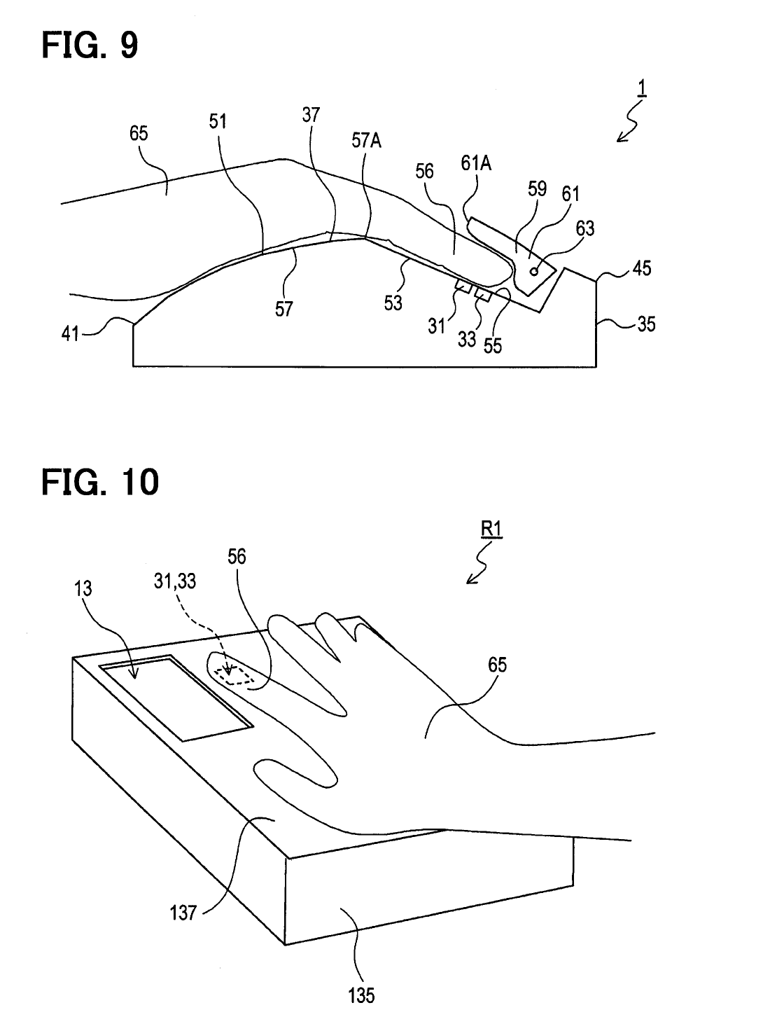

The second region 51 has the finger fitting groove 53 which is lower than the circumstance by one step. The finger fitting groove 53 corresponds to a measurement target finger placement region. The finger fitting groove 53 is a groove in a rectangular shape when viewed from above. The finger fitting groove 53 is located at a position in the second region 51 close to the side 45 in the longitudinal direction da and at a position in the second region 51 close to the boundary line 47 in the lateral direction db. The longitudinal direction of the finger fitting groove 53 is parallel to the longitudinal direction da. As shown in FIGS. 7 and 9, a bottom surface 55 of the finger fitting groove 53 is inclined so that the bottom surface 55 becomes lower on the side of the side 45. As shown in FIG. 8, a width of the finger fitting groove 53 is configured to allow the measurement target finger 56 to be inserted into the finger fitting groove 53.

A region on the upper surface 37 which is closer to the side 41 than the finger fitting groove 53 is defined as a palm placement region 57. The bottom surface 55 is lower than an end portion 57A of the palm placement region 57 on the side of the finger fitting groove 53. The housing 35 includes a pressing unit 59 at a position of the finger fitting groove 53 on the side of the side 45. The pressing unit 59 is configured to pivotally support a rectangular plate-shaped pressing piece 61 with a shaft 63. The longitudinal direction of the pressing piece 61 is parallel to the longitudinal direction da. The shaft 63 pivotally supports a portion of the pressing piece 61 on the side of the side 45. An axial direction of the shaft 63 is parallel to the lateral direction db. The pressing piece 61 is rotatable about the shaft 63 in directions Ra-Rb in FIG. 7.

When the pressing piece 61 rotates in the direction Ra, an end portion 61A of the pressing piece 61 on a side opposite to the shaft 63 comes closer to the bottom surface 55. Further, when the pressing piece 61 rotates in the direction Rb, the end portion 61A moves away from the bottom surface 55. The pressing piece 61 is urged by a spring not shown so as to rotate in the direction Ra.

As shown in FIGS. 7 and 9, the light emitting diode 31 and the photodiode 33 are provided on the bottom surface 55. The photodiode 33 receives a light reflected from the measurement target finger 56, which has been inserted into the finger fitting groove 53, out of the light irradiated with the light emitting diode 31, and takes out the received light as an electric signal.

As shown in FIGS. 8 and 9, a palm 65 of the subject is placed on the palm placement region 57. Further, the measurement target finger 56 is inserted into the finger fitting groove 53. At this time, the longitudinal direction of the palm 65 is parallel to the longitudinal direction da, and the lateral direction of the palm 65 is parallel to the lateral direction db. In this example, the longitudinal direction of the palm 65 represents a direction from a center of a wrist of the subject to a base of a middle finger. In addition, the lateral direction of the palm 65 represents a direction orthogonal to the longitudinal direction of the palm 65 described above and along a plane of the palm.

As described above, the second region 51 is in a shape convex upward with respect to the longitudinal direction da. Since the longitudinal direction of the palm 65 is parallel to the longitudinal direction da, the second region 51 is in a shape convex upward with respect to the longitudinal direction of the palm 65. The palm placement region 57, which is a part of the second region 51, is in a shape convex upward with respect to the longitudinal direction of the palm 65.

Further, as described above, the second region 51 is inclined so that the second region 51 on the side of the boundary line 47 in the lateral direction db is higher and the second region 51 on the side of the side 43 in the lateral direction db is lower. The lateral direction of the palm 65 is parallel to the lateral direction db. The side of the side 43 corresponds to a direction toward a little finger in the palm 65. Therefore, the second region 51 is inclined such that the second region 51 becomes lower on the side of the little finger. The palm placement region 57, which is a part of the second region 51, is also inclined such that the palm placement region 57 becomes lower on the side of the little finger.

The pressing piece 61 presses a tip portion of the measurement target finger 56 which has been inserted into the finger fitting groove 53 in the direction toward the bottom surface 55. The light emitting diode 31 and the photodiode 33 are located so as to sandwich the tip portion of the measurement target finger 56 from above and below.

As shown in FIG. 8, a thumb 67 of the subject is placed on a region of the first region 49 close to the boundary line 47 (hereinafter referred to as the thumb placement region 69). The thumb placement region 69 is lower than the end portion 57 B of the palm placement region 57 on the side of the thumb 67.

2. Processing to be Executed by the Blood Pressure Measurement Device 1

(1) Cuffless Measurement

When the cuffless measurement switch 7 is operated, the blood pressure measurement device 1 implements a cuffless measurement. In the cuffless measurement, the control unit 3 firstly acquires a pulse wave signal by using the pulse wave sensor 5 for a predetermined period. Subsequently, the control unit 3 measures a blood pressure of the subject based on the acquired pulse wave signal. The method of measuring the blood pressure based on the pulse wave signal can be appropriately selected from well-known methods (for example, the methods disclosed in Publication of Japanese Utility Model Application No. H7-9305 and Publication of unexamined Japanese Patent Application No. H7-308295).

Subsequently, the control unit 3 calibrates the blood pressure. The calibration of the blood pressure is a process of adding a calibration value to the blood pressure measured as described above. The calibration value is a value obtained by simultaneously implementing the cuff measurement and the cuffless measurement for the same subject and subtracting the latter measurement value from the former measurement value. The calibration value is determined by a calibration process to be described later and is stored in the memory 29. The blood pressure measurement device 1 indicates the calibrated measurement value on the display unit 13.

(2) Cuff Measurement

When the cuff measurement switch 9 is operated, the blood pressure measurement device 1 implements a cuff measurement as follows. First, the control unit 3 sets a pressure in the cuff 25 to a target compression pressure of about 180 mmHg with the use of the pump drive circuit 15, the valve drive circuit 17, the pump 21, and the valve 23. Subsequently, the control unit 3 reduces the pressure inside the cuff 25 at a gentle speed of about 3 mmHg/sec with the use of the pump drive circuit 15, the valve drive circuit 17, the pump 21, and the valve 23.

The control unit 3 calculates a systolic blood pressure value, a mean blood pressure value, and a diastolic blood pressure value according to an oscillometric system blood pressure value determination program based on a change in an amplitude of the signal obtained by the pressure sensor 19 in the course of reducing the pressure. The blood pressure measurement device 1 indicates a measurement value of the cuff measurement on the display unit 13.

(3) Calibration Process

When the calibration switch 11 is operated, the blood pressure measurement device 1 implements a calibration process. The calibration process is a process of implementing the cuff measurement and the cuffless measurement at the same time and storing a value obtained by subtracting the latter measured value from the former measurement value in the memory 29 as a calibration value. After the calibration process, the control unit 3 indicates the measurement value of the cuff measurement on the display unit 13.

3. Effects of the Blood Pressure Measurement Device 1

(1A) The shape of the palm placement region 57 has a shape convex upward with respect to the longitudinal direction of the palm 65 and a shape inclined so that the palm placement region 57 is lowered on the side of the little finger in the lateral direction of the palm 65. For that reason, when the cuffless measurement is implemented, the measurement target finger 56 hardly strains with an unnecessary force, and the measurement target finger 56 is in a relaxed state. As a result, the noise in the pulse wave signal is reduced, and the S/N of the pulse wave signal is improved.

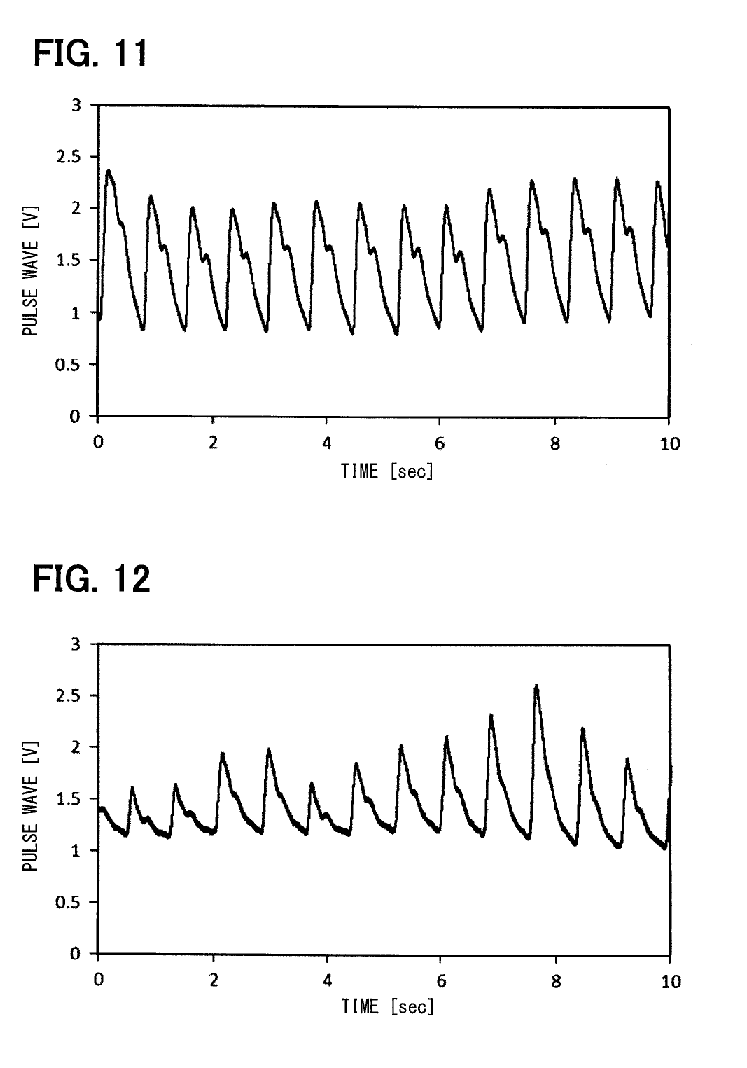

Experiments have been conducted to ascertain the above effects. A pulse wave signal is acquired by using the blood pressure measurement device 1. The results are illustrated in FIG. 11. An average value of a pulse wave amplitude is 1.37 V, and a standard deviation of the pulse wave amplitude is 0.12 V.

On the other hand, as a comparative example, as shown in FIG. 10, a blood pressure measurement device R1, which is the same in a basic configuration as the blood pressure measurement device 1 but in which an upper surface 137 of the housing 135 is all flat, is prepared. The light emitting diode 31 and the photodiode 33 of the pulse wave sensor and the display unit 13 are provided on a surface of the upper surface 137.

The palm 65 is placed on the upper surface 137 and the pulse wave signal is acquired by using the blood pressure measurement device R1. The results are illustrated in FIG. 12. The average value of the pulse wave amplitude is 0.65 V, and the standard deviation of the pulse wave amplitude is 0.27 V. The standard deviation of the pulse wave amplitude in the case of using the blood pressure measurement device R1 is remarkably large as compared with the case of using the blood pressure measurement device 1. From the experimental results, the above effects are confirmed.

(1B) The finger fitting groove 53 is lower than the end portion 57A. For that reason, when the cuffless measurement is implemented, the measurement target finger 56 is in a more relaxed state. As a result, an S/N in the pulse wave signal is further improved.

(1C) The blood pressure measurement device 1 further includes the pressing unit 59 that presses the measurement target finger 56 in the direction toward the bottom surface 55. For that reason, a contact state between the measurement target finger 56, and the light emitting diode 31 and the photodiode 33 is further stabilized. As a result, an S/N in the pulse wave signal is further improved.

(1D) The finger fitting groove 53 is inclined so as to be lower at the tip end of the measurement target finger 56. For that reason, when the cuffless measurement is implemented, the measurement target finger 56 is in a more relaxed state. As a result, an S/N in the pulse wave signal is further improved.

(1E) The thumb placement region 69 is lower than an end portion 57B. For that reason, when the cuffless measurement is implemented, the measurement target finger 56 is in a more relaxed state. As a result, an S/N in the pulse wave signal is further improved.

### OTHER EMBODIMENTS

The embodiments for carrying out the present disclosure have been described above. However, the present disclosure is not limited to the above-described embodiments, but can be variously modified.

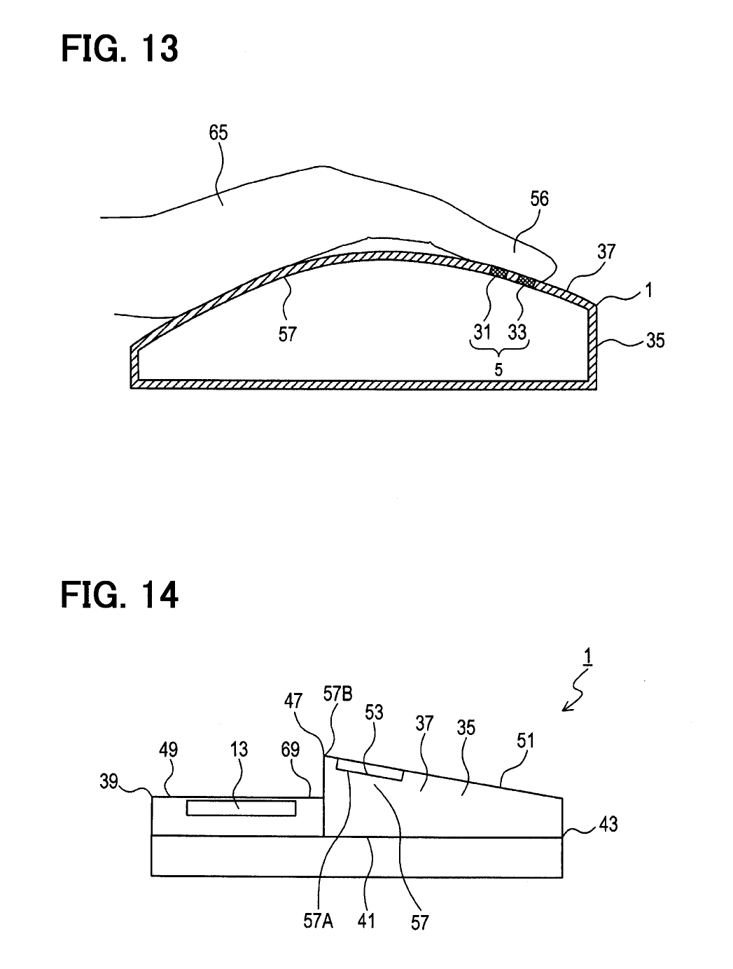

(1) A shape of the upper surface 37 can be appropriately determined. For example, as shown in FIG. 13, in a side cross section parallel to the longitudinal direction da, the palm placement region 57 and the region where the measurement target finger 56 is placed may have a continuous curved shape. Further, as shown in FIG. 14, in a side cross section parallel to the lateral direction db, the second region 51 may be flat.

(2) As shown in FIG. 13, the light emitting diode 31 and the photodiode 33 may be provided on the upper surface 37. In this case, the photodiode 33 receives the light reflected in a capillary blood vessel of the measurement target finger 56, and detects the pulse wave signal. In addition, the light emitting diode 31 and the photodiode 33 may be provided on the pressing piece 61. Also in this case, the photodiode 33 receives the light reflected in the capillary blood vessel of the measurement target finger 56 and detects the pulse wave signal.

(3) The measurement target finger 56 may be any one of the index finger, the middle finger, a ring finger, the little finger, and the thumb.

(4) The pulse wave sensor 5 may have another light source such as a laser light source instead of the light emitting diode 31. In addition, the pulse wave sensor 5 may have another photodetector such as a phototransistor or a CCD instead of the photodiode 33.

(5) The blood pressure measurement device 1 may not include the pressing unit 59.

(6) The thumb placement region 69 may be at the same height as that of the end portion 57B.

(7) The blood pressure measurement device 1 may be provided with a placement table on which the palm 65 can be placed, separately from the housing 35. The shape of the placement table can be the same as the shape of the housing 35 described above. Further, the pulse wave sensor 5 can be provided on the placing table, and the pulse wave signal can be acquired from the measurement target finger 56.

(8) The blood pressure measurement device 1 may be provided with a sensor for detecting insertion of the measurement target finger 56 into the finger fitting groove 53. The blood pressure measurement device 1 may automatically start the cuffless measurement or the calibration process when the insertion of the measurement target finger 56 is detected with the sensor.

Examples of the sensor described above include a mechanical sensor, a sensor for detecting a light, a sensor for detecting a temperature, and the like. The mechanical sensor can detect the insertion of the measurement target finger 56, for example, by the rotation of the pressing piece 61 when the measurement target finger 56 is inserted into the finger fitting groove 53.

Further, the mechanical sensor may be configured with a sensor that detects that the measurement target finger 56 presses a part or the whole of the pulse wave sensor 5. As the sensor for detecting the light, there is, for example, a sensor for detecting that the light incident on the photodiode 33 changes due to insertion of the measurement target finger 56. As the sensor for detecting the temperature, there is, for example, a sensor that is provided in the housing 35 for detecting a change in the temperature when the measurement target finger 56 is inserted.

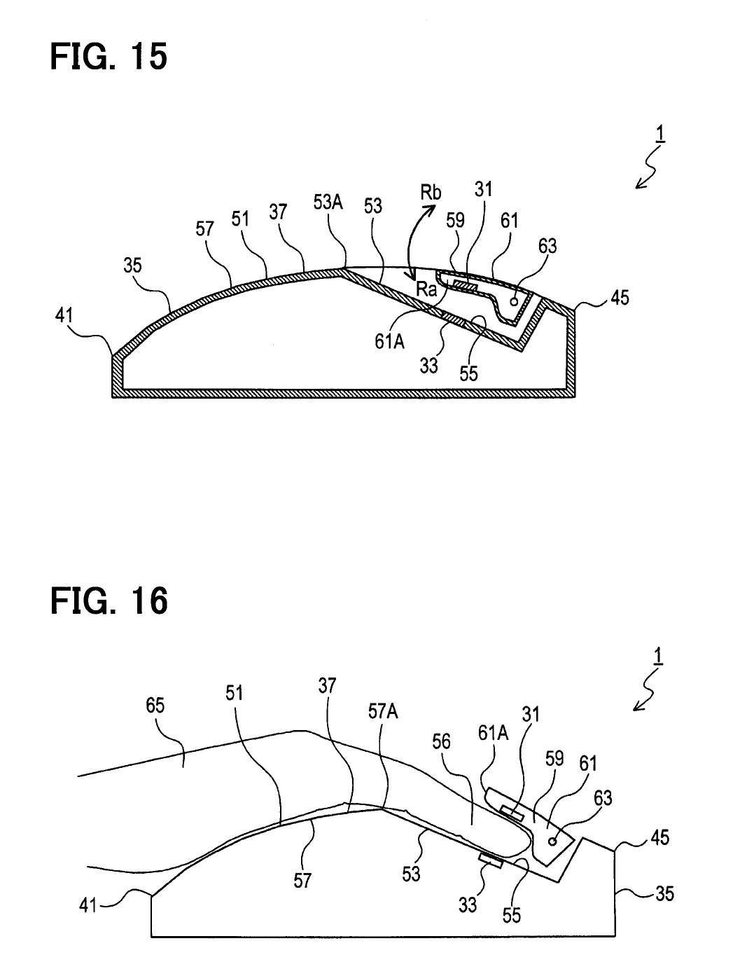

(9) In the first embodiment, as shown in FIGS. 15 and 16, the photodiode 33 may be provided on the bottom surface 55, and the light emitting diode 31 may be provided at the position on the side of the end portion 61A of the pressing piece 61. The photodiode 33 receives a light transmitted through the measurement target finger 56 which has been inserted into the finger fitting groove 53, out of the light irradiated with the light emitting diode 31, and takes out the received light as an electric signal. In this case, the light emitted with the light emitting diode 31 is a near infrared light having a wavelength of 7000 Å or more.

(10) A function of one constituent element in the above-described embodiments may be distributed to a plurality of constituent elements, or functions of a plurality of constituent elements may be integrated into one constituent element. A part of the configuration according to the above-described embodiment may be omitted. In addition, at least a part of the configuration in the above embodiments may be added to or replaced with another configuration in the above embodiments. In addition, all aspects that are included in the technical spirit that is specified in the attached claims are embodiments of the present disclosure.

(11) The present disclosure can be produced with various configurations such as a system having the blood pressure measurement device as a component, a program for causing a computer to function as a control unit of the blood pressure measurement device, a non-transitory tangible storage medium storing the program such as a semiconductor memory, or a method for measuring a blood pressure in addition to the blood pressure measurement device described above.

The blood pressure measurement device 1 described above includes the pulse wave sensor 5 to detect the pulse wave signal from the measurement target finger, which is a part of the fingers of the subject; the placement table 35 to enable the palm of the subject to be placed thereon; and the blood pressure measurement unit 3 to measure the blood pressure based on the pulse wave signal detected with the pulse wave sensor. The upper surface 37 of the placement table has a palm placement region 57. The palm placement region is in a shape convex upward with respect to the longitudinal direction of the palm. The palm placement region is inclined such that the palm placement region is lowered on the side of the little finger in the lateral direction of the palm. With the configuration described above, noise in the pulse wave signal can be reduced.

The present disclosure has been described with reference to the examples, but the present disclosure is not limited to the examples or the structures. The present disclosure includes various modification examples and modifications within the same range. In addition, it should be understood that various combinations or aspects, or other combinations or aspects, in which only one element, one or more elements, or one or less elements is included to the various combinations or aspects, are included in the scope or the technical idea of the present disclosure.

1. A blood pressure measurement device comprising:
a pulse wave sensor to detect a pulse wave signal from a measurement target finger, which is a part of fingers of a subject;
a placement table to enable a palm of the subject to be placed thereon; and
a blood pressure measurement unit to measure a blood pressure based on the pulse wave signal detected with the pulse wave sensor, wherein
an upper surface of the placement table has a palm placement region in a shape, which is convex upward with respect to a longitudinal direction of the palm and which is inclined to lower on a side of a little finger in a lateral direction of the palm.

2. The blood pressure measurement device according to claim 1, wherein
the upper surface further includes a measurement target finger placement region, which is lower than an end portion of the palm placement region on a side of the measurement target finger and which enables the measurement target finger to be placed thereon.

3. The blood pressure measurement device according to claim 2, further comprising:
a pressing unit to press the measurement target finger in a direction toward the measurement target finger placement region.

4. The blood pressure measurement device according to claim 2, wherein
the measurement target finger placement region has a slope to lower on a side of a distal end of the measurement target finger.

5. The blood pressure measurement device according to claim 1, wherein
the upper surface further includes a thumb placement region, which is lower than an end portion of the palm placement region on a side of a thumb and which enables the thumb of the subject to be placed thereon.
